(19) United States
(12) Patent Application Publication
(10) Pub. No.: US 2025/0339244 A1
(43) Pub. Date: Nov. 6, 2025

(54) METHOD FOR ACQUIRING A MODEL OF A DENTAL ARCH

(71) Applicant: DENTAL MONITORING, PARIS (FR)

(72) Inventors: Thomas PELLISSARD, PARIS (FR); Guillaume GHYSELINCK, CANTIN (FR)

(21) Appl. No.: 18/868,543

(22) PCT Filed: May 23, 2023
(86) PCT No.: PCT/EP2023/063808

§ 371 Date: Nov. 22, 2024

(30) Foreign Application Priority Data
May 24, 2022 (WO) PCT/EP2022/064127
Jun. 23, 2022 (FR) FR2206233

(51) Int. Cl. A61C 9/00; A61B 1/24; A61B 1/32; A61C 13/34

(52) U.S. Cl. CPC A61C 9/0053; A61B 1/24; A61B 1/32; A61C 13/34

(57) ABSTRACT

A method for acquiring at least one image of at least one dental arch of a user (U) by means of a mobile telephone (12′) and an acquisition tool (31′) comprising an acquisition head (32′) provided with a camera (33′), wherein method the acquisition head: —acquires said image and transmits it to the mobile telephone, or —acquires a signal and transfers the signal to the mobile telephone in order that said mobile telephone generates the image from the signal, autonomously or with the aid of a computer with which said mobile telephone is in communication, the method including, after said acquisition step, an analysis of said image so as to define the dental situation of the user and/or to check the proper implementation of an ongoing active or passive orthodontic treatment.

### TECHNICAL FIELD

The present invention concerns a method for acquiring a model of a user's dental arch and a computer program for implementing this method.

### STATE OF THE ART

It is desirable for everyone to have their teeth checked regularly, in particular to ensure that the position and/or shape and/or appearance (or “texture”) of their teeth are not changing unfavorably.

In the case of orthodontic treatment, this unfavorable trend may lead to a change in treatment. After orthodontic treatment, this unfavorable trend, known as “recurrence”, may lead to the need for renewed treatment. Finally, in a more general way and independently of any treatment, everyone may wish to monitor any movements and/or changes in the shape and/or appearance of their teeth.

Traditionally, the checks are carried out by an orthodontist or dentist, who are the only ones with the right equipment. These checks are therefore costly. Furthermore, the visits are burdensome. Finally, the professional scanners available are accurate, but require special skills. They are typically used on the patient, for intraoral acquisition, or on a cast of the patient's arches, for extraoral acquisition.

In addition, U.S. Pat. No. 15/522,520 describes a method which, based on a simple photograph of the teeth taken by the user at an updated instant, enables the accurate assessment of the movement and/or deformation of the teeth since an initial instant. To this end, a digital three-dimensional model of the user's dental arch is created, preferably using a professional scanner. This initial model is then cut to define a tooth model for each tooth. Finally, the tooth models are moved to transform the initial dental arch model to match the photograph as closely as possible. This method produces a model of the current arch with excellent accuracy, without the user having to go to the dentist for a scan of their teeth. This model can then be compared with the initial model to check the positioning and/or shape of the user's teeth.

This method is convenient for the user, but requires at least one appointment to acquire the initial arch model. It then requires heavy computer processing to break down the initial model, then deform it.

There is therefore a need for a method of monitoring a user's dental situation remotely, as described in U.S. Pat. No. 15/522,520, but which is even more convenient for the user and quicker to implement.

One objective of the present invention is to address this problem, at least partially.

### SUMMARY OF THE INVENTION

The invention provides a method for acquiring a model of at least one dental arch of a user, said method comprising the following steps:
a) at an updated instant, acquisition, preferably extraoral, with a portable scanner, by the user, of a digital three-dimensional model of said arch, or “acquired model”, and optionally trimming the arch model so as to isolate a part of the arch model, preferably a tooth model,
in order to obtain an “updated model”, the updated model being the acquired model or the part of the acquired model isolated by cutting, the object represented by the updated model being called the “updated object”.

As will be seen in greater detail later in the description, the inventors have discovered that it is possible to use a portable scanner to produce, preferably extraorally and without special precautions, a model of an arch or tooth of sufficient quality to be used in orthodontics. Such a method seemed incompatible with the acquisition of a sufficiently complete and accurate model.

Advantageously, acquisition can be carried out by the user on their own, opening up a wide range of applications. In particular, acquisition no longer requires a trip to a dental professional. In addition, a method according to the invention enables the user's dental situation to be analyzed more quickly than with prior art methods. In particular, no construction of an arch model from photos is required.

In general, 3D models of dental arches are traditionally acquired intraorally, using an optical 3D scanner. Intraoral acquisition enables the sensor to be very close to the arch, and therefore to provide highly accurate information.

Extraoral (or “extrabuccal”) acquisition devices, that is, ones where the acquisition sensor, in particular the sensor of a camera or stills camera, is not inserted into the user's mouth, are a recent development, and use photos to deform an initial model obtained with a conventional optical 3D scanner. The computer processing required for this deformation is costly.

It is to the inventors' credit that they tested a portable, preferably extraoral, scanner, in particular a laser remote sensor, and discovered that such a scanner enables the patient to acquire a good-quality model of their dental arches. Advantageously, no initial model, for example acquired at the start of orthodontic treatment, needs to be acquired and then deformed from the images acquired by the scanner. By processing the images acquired by the scanner, a model of the dental arch can be obtained directly, following the techniques conventionally used for 3D optical scanners.

In an advantageous embodiment, the portable scanner is low-precision. All one needs to do is record the spatial position of a few noteworthy points on the arch to create an updated model. Advantageously, the acquisition of a low-precision model is possible with limited, portable technical means. A low-precision model also requires little memory for storage. It can be easily and quickly transmitted remotely, for example by radio.

Preferably, the portable scanner
is integrated into a mobile telephone for so-called extraoral acquisition, or
comprises a mobile telephone and an acquisition tool comprising an acquisition head that can be inserted into the user's mouth, which
acquires the acquired model, preferably using a laser remote sensor, and transmits it to the mobile telephone, or
acquires a signal and transfers it to the mobile telephone so that said mobile telephone generates the acquired model from said signal, either autonomously or with the help of a computer with which said mobile telephone is in communication.

Preferably, the mobile telephone transmits the acquired and/or updated model to a dental professional, preferably over the air, preferably at a distance greater than 100 m, or greater than 1 km, or greater than 10 km and/or less than 50,000 km from the user.

An analysis method according to the invention may further comprise one or more of the following optional features:
in step a), the updated model is processed by computer in order to correct it, with the correction potentially comprising modifying the updated model or replacing the updated model with a correction model;
in step a), the updated model is compared with a correction model so as to obtain a measurement of a shape difference between the updated model and the correction model, then
the updated model is modified so as to reduce said shape difference, preferably so as to minimize said shape difference, preferably by means of a metaheuristic method, in particular selected from the methods listed below, preferably by simulated annealing, or
depending on said measurement, the updated modulus is left unchanged or the updated model is replaced by the correction model;
in step a), the updated model is submitted to a neural network trained to render more realistic a digital three-dimensional model presented to it as input;
the updated object is said user's arch or a tooth of said arch;
the correction model is a model
obtained, at an instant different from the updated instant, by scanning said updated object, or
representing said object updated with a theoretical shape, preferably resulting from a simulation, or
a model of an object representative of a set of individuals, said object being of the same type as the updated object, preferably an arch or a tooth, for example a typodont or a tooth resulting from a typodont;
the correction model is:
a model of the updated object obtained by a scan, preferably with the portable scanner or with a professional scanner, preferably at a time more than 2 weeks, 4 weeks, 6 weeks, 2 months, 3 months and/or less than 12 months, or less than 6 months before the updated instant, or
a model of the updated object, which simulates the shape of said updated object as anticipated for the updated instant and which, preferably, was realized at an instant more than 2 weeks, 4 weeks, 6 weeks, 2 months, 3 months and/or less than 6 months before the updated instant, or
a model of the updated object, which simulates the shape of said updated object as anticipated for a “correction” instant, later or earlier than the updated instant, the time interval between the updated and correction instants preferably being greater than one week, more preferably greater than 2 weeks, 4 weeks, 6 weeks, 2 months and/or less than 6 months, said model having preferably been produced more than 2 weeks, more than 4 weeks, more than 6 weeks, more than 2 months or more than 3 months before the updated instant, or
a historical model chosen from a historical library comprising more than 1,000 historical models representing objects of the same type as the updated model, said choice preferably being guided in such a way that the historical model chosen is the historical model having the nearest shape to the updated model, or
a model obtained by statistical processing of the historical models in said historical library, preferably in such a way that the model obtained by statistical processing is representative of a population of individuals;
the historical library comprises only historical models that meet the same classification criteria as the updated model, for example relating to individuals with at least one characteristic in common with the user, for example the same age and/or the same sex and/or the same pathology and/or undergoing the same or similar orthodontic treatment;
in step a), the updated model is corrected by inputting the updated model to the input of a neural network trained to correct models, preferably chosen from the neural networks listed in the detailed description of step iv) below; and/or
in step a), the updated model is corrected in the following steps:
i) creating a historical library comprising more than 1,000, preferably more than 5,000, more preferably more than 10,000 historical models, each historical model modeling an object of the same type as the updated object, for example modeling an arch or a tooth if the updated model is modeling an arch or a tooth, respectively, and
assigning a value for a classification criterion to each historical model;
ii) analyzing the updated model, in order to determine the value of said classification criterion for the updated object;
iii) searching the historical library for a historical model having the same value for said classification criterion and presenting maximum proximity to said updated model, or “optimal model”;
iv) modifying the updated model based on information about the optimal model, which may comprise replacing the updated model with the optimal model;
in step a), the acquired model is broken down so as to define a plurality of tooth models, then for each tooth model considered to be an updated model, a cycle of steps i) to iv) is carried out wherein the optimal model determined in step iv) is arranged so as to replace said tooth model in the acquired model, which advantageously makes it possible to reconstitute a high-precision arch model from an acquired low-precision model;
in step a), the updated model is corrected in the following steps:
i′) defining:
a first certain zone made up of the points of the updated model representing a part of the patient, for example a tooth, with an accuracy greater than 90%, preferably greater than 95%, more preferably greater than 99% or “first certain points”, and
a first uncertain zone, constituting the 100% complement of the updated model;
ii′) extrapolation of the first certain zone, from the first certain zone only, to define, in the region of the first uncertain zone, a first reconstructed zone, then defining:
a second certain zone made up of the points of the first uncertain zone separated from the first reconstructed zone by a distance less than a threshold distance, or “second certain points”; and
a second uncertain zone, constituting the 100% complement of the first uncertain zone;
iii′) extrapolating the set consisting of the first and second certain zones, from the single set, to define, in the region of the second uncertain zone, a second reconstructed zone, then
replacing the second uncertain zone with the second reconstructed zone, so as to obtain a cleaned updated model;
in step a), the updated model is corrected by subjecting the updated model to a neural network trained by supplying it as input with raw models of objects of the same type as the updated object, and as output with said raw models rendered hyperrealistic;
in step a), the updated model is processed by computer to simplify it;
the portable scanner is integrated into a mobile telephone or comprises a mobile telephone and an acquisition tool comprising an acquisition head that can be inserted into the user's mouth, the acquisition tool being in communication with the mobile telephone to transmit the acquired model or the updated model;
the acquisition head is connected to the mobile telephone, preferably via Bluetooth® or cable;
the mobile telephone is used to transmit, by radio, the acquired or updated model, preferably to a dental professional, and in particular to an orthodontist, and/or to a data processing center, preferably for the implementation of steps b) and/or c);
the portable scanner is a laser remote sensor, called lidar for “light detection and ranging”;
in step a), the portable scanner projects structured light directly onto the patient's teeth and acquires images different from photographs;
in step a), the user modifies the angulation of the portable scanner, preferably by moving the portable scanner relative to the patient's teeth, preferably horizontally and/or vertically, preferably with the mouth open or closed;
in step a), the user spreads their lips and/or cheeks to make their teeth visible to the portable scanner, then acquires the acquired model, preferably extraorally, that is, without bringing the portable scanner even partially into their mouth;
preferably, the user uses a retractor and/or a portable scanner support to improve the quality of the acquired model;
in step a), the portable scanner is immobilized on a support having a rim, the rim being inserted between the lips and teeth of the user;
the support comprises a tubular spacer which defines an oral opening, said rim extending to the periphery of the oral opening;
in step a), the user modifies the angulation of the portable scanner, preferably by moving the support relative to the patient's teeth, preferably horizontally and/or vertically, preferably with the mouth open and the mouth closed, keeping the edge of the support between the user's teeth and the user's lips;
in step a), the model acquired with the portable scanner is broken down to define a plurality of tooth models, then each of said tooth models is successively corrected and/or simplified, preferably as described above;
the method comprises, after step a), the following step:

b) determining at least one value of a dimensional parameter of the updated model, or “dimensional value”, and/or of an appearance parameter of the updated model, or “appearance value”;
in step b), more than two dimensional values are defined, preferably enough dimensional values to define a position in space of at least one point of the updated model, preferably more than 10, more than 100, more than 500 points of the updated model;
the dimensional parameter is chosen from
a dimension of the updated model;
a distance of a noteworthy point of the updated model from a reference frame that is preferably fixed with respect to the updated model, preferably a reference model arranged, like the updated model, in a standardized configuration, and
a parameter derived from one or more dimensions of the updated model and/or from one or more distances of one or more noteworthy points of the updated model from said reference frame;
the appearance parameter is selected from color, reflectance, transparency, reflectivity, shade, translucency, opalescence, an index of the presence of tartar, dental plaque or food on the tooth;
to determine a said dimensional value, a distance is measured between a point of the updated model and a reference model arranged, like the updated model, in a standardized configuration;
the reference model is preferably
a model of the updated object obtained by a scan, preferably with the portable scanner or with a professional scanner, preferably at a time more than 2 weeks, 4 weeks, 6 weeks, 2 months, 3 months and/or less than 6 months before the updated instant, or
a model of the updated object, which simulates the shape of said object as anticipated for the updated instant and which, preferably, was realized at an instant more than 2 weeks, 4 weeks, 6 weeks, 2 months, 3 months and/or less than 6 months before the updated instant, or
a model of the updated object, which simulates the shape of said object as anticipated for a reference instant, later or earlier than the updated instant, the time interval between the updated and reference instants preferably being greater than one week, more preferably greater than 2 weeks, 4 weeks, 6 weeks, 2 months and/or less than 6 months,
said model having preferably been produced more than 2 weeks, more than 4 weeks, more than 6 weeks, more than 2 months or more than 3 months before the updated instant, or
a historical model chosen from a historical library comprising more than 1,000, preferably more than 10,000, preferably more than 100,000 historical models representing objects of the same type as the updated object, said choice preferably being guided in such a way that the historical model chosen is the historical model having the nearest shape to the updated model, or
a model obtained by statistical processing of the historical models in said historical library, preferably in such a way that the model obtained by statistical processing is representative of a population of individuals;
the method comprises, after step b), the following step:
c) using the dimensional value and/or appearance value to:
detect or evaluate a position or shape of a tooth and/or a change of a position or shape of a tooth and/or a rate of change of a position or shape of a tooth, and/or
detect or evaluate a position or shape of an orthodontic appliance and/or a change of a position or shape of an orthodontic appliance and/or a rate of change of a position or shape of an orthodontic appliance, and/or
measure changes in the shape of the patient's teeth between two dates, and/or
in a dental office;
in step c), the dimensional value and/or the appearance value are used to
detect or evaluate the position or shape of a stain or cavity:
monitor tooth eruption, and/or
detect recurrence or abnormal tooth position, and/or
detect tooth abrasion, and/or
track the opening or closing of at least one gap between two teeth, and/or
check the stability or modification of occlusion,
track the movement of a tooth to a predetermined position, and/or
detect or evaluate a detached orthodontic band or aligner,
optimize the appointment date with a dental professional, and/or
evaluate an orthodontic index, in particular selected from the orthodontic indices listed in the definition of an orthodontic index below, preferably an orthodontic index indicating whether
the user has reached occlusion class I for canines, and/or
the user has reached occlusion class I for molars, and/or
the patient's anterior spaces are closed, and/or
all spaces resulting from a tooth extraction are closed, and/or
the user has a normal overjet, preferably between 1 and 3 mm, and/or
the user has a normal overbite, preferably between 1 and 3 mm, and/or
the midlines of the upper and lower arches are shifted, and/or
the user has no lateral shift of the upper arch relative to the lower arch, and/or
during the last two checks, no tooth movement was detected, and/or
all the temporary teeth have fallen out, or an orthodontic index that quantitatively evaluates and/or assesses the temporal evolution of:
the occlusion class for canines, and/or
the occlusion class for molars, and/or
the patient's anterior spaces, and/or
spaces resulting from tooth extraction, and/or
overjet, and/or
overbite, and/or
shift between the midlines of the upper and lower arches, and/or
lateral shift of the upper arch relative to the lower arch, and/or
tooth movement during the last two checks, and/or
evaluate the effectiveness of active orthodontic treatment, and/or
measure the activity of an active orthodontic appliance; and/or
measure loss of efficiency of a passive orthodontic appliance; and/or
compare the positioning of the user's teeth, at the updated instant, with that of said teeth as represented by a theoretical target model, preferably an intermediate model representing said teeth in an anticipated position, according to a treatment plan, for a final stage or for an intermediate stage of orthodontic treatment; and/or
evaluate the need to correct or adapt orthodontic treatment, for example by designing and manufacturing a new series of orthodontic aligners as part of orthodontic treatment with orthodontic aligners, or by changing the type of orthodontic treatment, for example from brackets to aligners, or vice versa); and/or
measure changes in the shape of the patient's teeth between two dates separated by the occurrence of an impact on the teeth or by the application of a dental device intended for the treatment of sleep apnea, or by the occurrence of a graft in the patient's mouth;
in step a), the model acquired with the mobile telephone is broken down to define a plurality of tooth models, then a said step b) is performed to define at least one dimensional value for each tooth model, defined as the updated model for said step b);
in step a), the user acquires, preferably with the same mobile telephone, said acquired model and one or more updated images, preferably color photos, preferably in realistic colors, and
in step b), information relating to a dimension and/or the appearance of one or more objects, preferably teeth, represented on the updated image(s) is determined, and said information is then used to supplement and/or correct said dimensional value and/or said appearance value determined from the updated model;
in step a), the acquired model comprises fewer than 500 points.

The invention further relates to:
a computer program, and in particular a specialized mobile telephone application, comprising program code instructions for executing step a), and preferably step b), and preferably step c), when said program is executed by a computer,
a data medium on which such a program is recorded, for example a memory or a CD-ROM, and
a portable scanner, in particular incorporated in a mobile telephone, wherein such a program is loaded.

The invention thus relates to a portable scanner, preferably integrated into a mobile telephone, suitable for implementing the acquisition in step a), and preferably one or more of the correction and/or simplification processes described in the present description, and preferably step b), and more preferably step c).

### DEFINITIONS

The term “user” means any person for whom a method according to the invention is implemented, whether that person is ill or not, or undergoing an orthodontic treatment or not.

The term “dental care professional” refers to any person qualified to provide dental care, including in particular orthodontists and dentists.

An “orthodontic treatment” is all or part of a treatment designed to modify the shape of a dental arch (active orthodontic treatment) or to maintain the shape of a dental arch, in particular after the end of an active orthodontic treatment (passive orthodontic treatment).

Orthodontic indices are synthetic indicators of the shape and/or change of the shape of the dental arches. They can be specific to one or both arches (“inter-arch” indices). Examples include:
overbite, overjet, crowding, in particular the Nance index, deviation of the inter-incisal midlines, classes of canine and/or molar occlusion an irregularity index, in particular the Little index, anterior open bite, lateral open bite, posterior lingual crossbite, posterior buccal joint inversion, ideal arch length, presence or absence of interdental spaces, curve of Spee levelling index, presence of significant rotation, e.g. greater than 10°, on certain teeth,
as well as combinations of these indices and changes thereto. Examples of orthodontic indices are those used to define the American Board of Orthodontics “ABO Discrepancy Index”.

An “orthodontic appliance” is a device worn or intended to be worn by a user. Orthodontic appliances can be used for therapeutic or prophylactic treatment, as well as for aesthetic purposes. An orthodontic appliance can be, in particular, an arch and bracket appliance, or an orthodontic aligner, or an auxiliary appliance of the Carrière Motion type.

“Arch” or “dental arch” means all or part of a dental arch.

An “image” refers to a two-dimensional digital representation, such as a photograph or a frame from a video. An image is made up of pixels.

The term “model” means a three-dimensional digital model. A model is made up of a set of voxels. It typically comprises a mesh of points connected by line segments, that is, an assembly of triangles.

A “tooth model” is a three-dimensional digital model of a tooth. A dental arch model can be cut to define tooth models for at least some, preferably all, of the teeth represented in the arch model. Tooth models are therefore models within the arch model.

An “arch model” is a model representing at least part of a dental arch, preferably at least 2, preferably at least 3, most preferably at least 4 teeth.

A model, in particular a model of an arch or a tooth, is “hyperrealistic”

when the viewer has the impression of observing the modeled object itself. In particular, the colors of the model are those of the object being modeled.

A “raw” model means a model resulting from a scan, possibly corrected according to the invention, but whose color has not been modified to make it hyperrealistic.

The “type” of a modeled object, and of the updated object in particular, defines the nature of that object. In particular, the object can be of the “tooth” or “arch” or “gum” type. The object can also be a tooth subgroup, for example the incisor group or the group of teeth bearing one or more tooth numbers, or an arch subgroup, for example the upper arch.

A “classification criterion” is an attribute of a modeled object, in particular an arch or a tooth, that enables it to be classified. For example, the classification criterion may be an occlusion class, a range for a dimension (e.g. height, width, concavity, inter-canine distance, inter-premolar width, inter-molar width, arch length or arch sag, arch perimeter) of the modeled object, the age, sex, pathology or orthodontic treatment of the person owning the modeled object, an orthodontic index, in particular chosen from the orthodontic indices listed above, or a combination of these criteria.

In particular, the use of a classification criterion makes it possible to select modeled objects with similar or identical characteristics. Advantageously, it enables the creation of a learning base properly suited to the object that a neural network is intended to process. For example, if a neural network is intended to correct tooth models representing teeth with number 14, it is preferable to train it with a training base containing only records relating to number 14 teeth. The tooth number is then used as a classification criterion.

A “normalized configuration” is the positioning of a model, in space,

according to a predetermined orientation, with a predetermined scale. To compare the shape of two models representing an object, for example an arch or a tooth, the two models can be arranged in a standardized configuration. Standardization methods for arranging and sizing a model according to a standardized configuration are well known. One way of comparing the shape of two models is to use an Iterative Closest Point search algorithm (ICP, described at https://fr.wikipedia.org/wiki/Iterative_Closest_Point).

The “breakdown” of an arch model into “tooth models” is an operation that delimits and makes autonomous the tooth representations (tooth models) in the arch model. Computer tools are available to manipulate tooth models in an arch model. An example of software for manipulating tooth models and creating a treatment scenario is the program Treat, described at https://en.wikipedia.org/wiki/Clear_aligners#cite_note-invisalignsystem-10.

A “statistical treatment” is one which, when applied to a set of data, enables us to determine characteristics specific to this set, such as a mean, a standard deviation, or a median value. Statistical processing tools are well known to the person skilled in the art.

“Metaheuristic” methods are well-known optimization methods. In the context of the present invention, they are preferably selected from the group formed by:
evolutionary algorithms, preferably selected from among evolution strategies, genetic algorithms, differential evolution algorithms, estimation of distribution algorithms, artificial immune systems, Shuffled Complex Evolution path relinking, simulated annealing, ant colony algorithms, particle swarm optimization algorithms, Tabu search, and the GRASP method;
the kangaroo algorithm,
the Fletcher-Powell method,
the noise injection method,
stochastic tunneling,
random-restart hill climbing,
the cross-entropy method, and
hybrid methods between the above-mentioned metaheuristic methods.

A measurement of the difference, or distance, between two objects is

called a “match” or “fit”. A “best fit” is when this difference is minimal.

A “neural network” or “artificial neural network” is a set of algorithms well known to the person skilled in the art. To be operational, a neural network must be trained by a learning process called “deep learning”, from a training base.

A “learning base” is a database of computer records suitable for training a neural network. The quality of the analysis performed by the neural network depends directly on the number of records in the training database. Typically, the learning base comprises more than 1,000, preferably more than 10,000 records.

The training of a neural network is adapted to the aim pursued and does not pose any particular difficulty for the person skilled in the art. Training a neural network consists in confronting it with a training base containing information on first and second objects, which the neural network must learn to “match”, that is, connect to each other.

Training can be based on a “paired” learning base, made up of “paired” records, that is, each comprising a first object for input to the neural network, and a corresponding second object for output from the neural network. We also say that the input and output of the neural network are “paired”. Training the neural network with all of these pairs teaches it to provide, from an object similar to the first objects, a corresponding object similar to the second objects.

The article “Image-to-Image Translation with Conditional Adversarial Networks” by Phillip Isola Jun-Yan Zhu, Tinghui Zhou, Alexei A. Efros, Berkeley AI Research (B AIR) Laboratory, UC Berkeley, shows the use of a paired learning base.

The function of a “reference frame” is to serve as a basis for measuring one or more distances. A reference frame can be, for example, a three-dimensional, orthonormal reference frame. The three-dimensional reference frame is preferably fixed relative to the model in question. If the model represents an arch, for example, it can originate from the center of the user's oral cavity. In particular, the three-dimensional reference frame is preferably independent of the position and orientation of the portable scanner.

The dimensions (length, width, height) of an arch are conventionally measured with the arch in a horizontal plane. The height direction Y is then the vertical direction. The width direction X is the transverse direction for the user, extending from the right to the left of the user. The length direction Z is the depth direction for the user, extending from the front to the back of the user.

The dimensions (length, width, height) of a tooth are conventionally measured with the arch in a horizontal plane. The height direction Y′ is then the vertical direction. The width direction X′ is the direction of the tooth's largest dimension when viewed from the front, perpendicular to the height direction. The length direction Z′ is perpendicular to the directions Y′ and X′.

According to the international convention of the FDI World Dental Federation, each tooth in a dental arch has a predetermined number. The tooth numbers defined by this convention are shown in FIG. 6.

A “noteworthy point” is a point on an arch or tooth model that can be identified, e.g. the apex of the tooth or at the tip of a cusp, a point of interdental contact, that is, of a tooth with an adjacent tooth, e.g. a mesial or distal point of the incisal edge of a tooth, or a point at the center of the tooth crown, or “barycenter”.

An “angulation” is an orientation of the optical axis of the portable scanner relative to the user, during model acquisition in step a).

A 3D scanner, or “scanner”, is a device that produces a model of a tooth or dental arch. Traditionally, it uses structured light to create a 3D model from different images, preferably by matching specific points on these images.

More specifically, the portable scanner projects structured light onto the patient's teeth while acquiring said images. The scanner can project a light pattern onto the teeth. The distortion of this pattern allows the spatial interpretation of the scene.

Traditional techniques include 1-dimensional or 2-dimensional pattern projection, multistripe laser triangulation (MLT), digital fringing, and phase modulation.

Alternatively or in addition to the structured light projection, the portable scanner projects modulated light onto the patient's teeth while acquiring said images. The projected light then changes, and the scanner's camera measures the variation in reflected light over time to deduce the distance it travels. Among the techniques conventionally used, the phase-modulated technique is particularly noteworthy.

Image analysis is used to build the model.

The images can be of the same type as those acquired by conventional intraoral 3D optical scanners.

The images are representations of the observed scene, in this case the patient's teeth, but their nature is specific to the nature of the light source illuminating the scene. Preferably, the images are not realistic representations of the scene, as a person would observe it directly.

The maximum difference in shape between the model acquired with the scanner and the scanned, full-scale object is inversely proportional to the scanner's performance. This is called the scanner's “acquisition resolution” or “precision”. The smaller the resolution, the more faithful the model is to reality.

A laser remote sensor is particularly well-suited to the invention, as it enables extraoral acquisition of a precise model of the arch, by the patient on their own, with the laser light projected directly onto the patient's teeth.

A professional scanner preferably has an accuracy of less than 5/10 mm (that is, the maximum difference in shape between the model acquired with the scanner and the actual object scanned, at true scale, is less than 5/10 mm), preferably less than 3/10 mm, preferably less than 1/10 mm, preferably less than 1/50 mm, more preferably less than 1/100 mm and/or greater than 1/500 mm.

A “mobile telephone” or “cellular telephone” is a device like the iPhone®. Such a device typically weighs less than 500 g or less than 200 g, and is equipped with a camera comprising a lens to take videos or photos, or even a scanner to acquire three-dimensional digital models. A mobile telephone is also capable of exchanging data with another device more than 500 km away from the mobile telephone, and is able to display on a screen the videos, photos or models it has acquired.

A retractor (or dental retractor) is a device used to pull back the lips. It comprises an upper and a lower flange, and/or a right and a left flange, extending around a retractor opening and intended to be inserted between the teeth and the lips. In the operating position, the user's lips rest on these edges, so that the teeth are visible through the retractor opening. A retractor thus makes it possible to observe the teeth without being obstructed by the lips.

However, the teeth do not rest on the retractor, so that by turning the head relative to the retractor, the user can change the teeth that are visible through the retractor opening. The user can also change the spacing between their dental arches. In particular, a retractor does not press on the teeth to spread the two jaws apart, but rather on the lips.

In one embodiment, a retractor is configured to elastically spread the upper and lower lips apart to expose the teeth visible through the retractor opening.

In one embodiment, a retractor is configured so that the distance between the top edge and the bottom edge, and/or between the right edge and the left edge, is constant.

Retractor are described, for example, in PCT/EP2015/074896, U.S. Pat. No. 6,923,761, or US 2004/0209225.

The “service position” is the position wherein the user acquires the model acquired in step a). When using a support to rigidly secure the portable scanner, the support is partially inserted into the user's mouth, as shown in FIGS. 2 and 3.

The “mouth closed” position is the occlusion position wherein the teeth of the patient's upper and lower arches are in contact. A “mouth open” position is one wherein the teeth of the patient's upper and lower arches are not in contact.

The method (excluding the acquisition operation with the portable scanner) according to the invention is implemented by computer, preferably exclusively by computer.

By “computer” we mean a computer processing unit, which includes a set of several machines with computer processing capabilities. In particular, this unit can be integrated into the portable scanner, or in a mobile telephone incorporating the portable scanner, or be a PC-type computer or server, for example a server remote from the user, e.g. being the “cloud” or a computer located at a dental professional's office. In such a case, mobile telephone and the computer comprise communication means for exchanging information with each other, in particular for transmitting the updated, optionally corrected and/or simplified model, and/or one or more dimensional values determined according to the invention.

Typically, a computer comprises a processor, a memory, a human-machine interface, typically comprising a screen, and a communication module via the Internet, WIFI, Bluetooth® or the telephone network. Software configured to implement a method of the invention is loaded into the computer's memory. The computer can also be connected to a printer.

“First” and “second” are used for the sake of clarity.

Similarly, for the sake of clarity:
the term “basic” refers to a model used in the preferred simplification method;
the term “reference” refers to a model used in step b) to evaluate a dimensional value or an appearance value, or to an instant at which the object modeled by the reference model is expected to have the shape or appearance of this model;
the term “correction” refers to a model or instant used in a preferred correction method;
the term “updated” refers to step a), and in particular to the model resulting from step a);
the term “historical” refers to one or more models acquired prior to the updated instant, in particular modeling an arch or a tooth of a “historical” person different from the user;
“optimal” refers to a model which, among a set of models, has the shape closest to the updated model.

“Vertical”, “horizontal”, “right”, “left”, “in front” or “from the front”, “behind”, “above”, “below” refer to a user standing vertically.

Unless otherwise indicated, “including” or “comprising” or “having” should be interpreted in a non-restrictive manner.

### BRIEF DESCRIPTION OF THE FIGURES

Further features and advantages of the invention will become apparent from the following detailed description and from an examination of the appended drawing, wherein:

FIG. 1 schematically shows an example of a kit according to the invention;

FIG. 2 schematically shows the kit according to the invention in a service position, with the user viewed from the front;

FIG. 3 schematically shows the kit according to the invention in a service position, with the user viewed from the side;

FIG. 4 shows a model acquired at three different acquisition resolutions;

FIG. 5 is an example of an acquired model, after processing to break down the tooth models; an example of a tooth model is colored dark grey;

FIG. 6 shows the tooth numbering used in dentistry;

FIG. 7 shows an acquisition method according to the invention;

FIG. 8 shows a first correction method according to the invention;

FIG. 9 shows a second correction method according to the invention;

FIG. 10 schematically shows an example of a portable scanner in one embodiment of the invention;

FIG. 11 shows a number of images that provide additional data;

FIG. 12 schematically shows an example of a device for implementing an image acquisition method according to the invention.

In the various figures, identical references are used to designate similar or identical objects.

### DETAILED DESCRIPTION

The aim of a method according to the invention, shown in FIG. 7, is to rapidly provide a digital three-dimensional model of a user's arch, or part of it, that is, an “updated model”.

In step a), at an updated instant, the user generates the “acquired model” using a portable scanner 6.

Preferably, the acquired model represents at least 2, preferably at least 3, more preferably at least 4 teeth, preferably all the teeth in the arch.

A portable scanner is an autonomous scanner, in particular in that it integrates its own power source, typically a battery, and in that its weight allows it to be handled by hand.

Preferably, the portable scanner weighs less than 1 kg, preferably less than 500 g, more preferably less than 200 g, and/or more than 50 g.

Preferably, the largest dimension of the portable scanner is less than 30 cm, 20 cm or 15 cm and/or greater than 5 cm.

The portable scanner preferably has an acquisition resolution of less than 10 mm, preferably less than 5 mm, preferably less than 3 mm, preferably less than 2 mm, preferably less than 1 mm, preferably less than 1/2 mm, preferably less than 1/5 mm, preferably less than 1/10 mm.

The portable scanner is preferably configured so that the acquired model comprises more than 5,000 and/or less than 200,000, or less than 150,000 points.

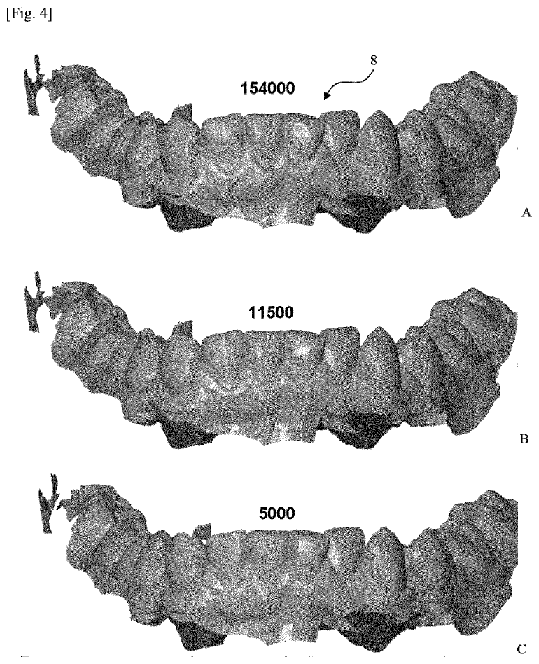

FIG. 4 shows examples of arch models 8 acquired with a portable scanner featuring 5,000, 11,500 and 154,000 points, respectively.

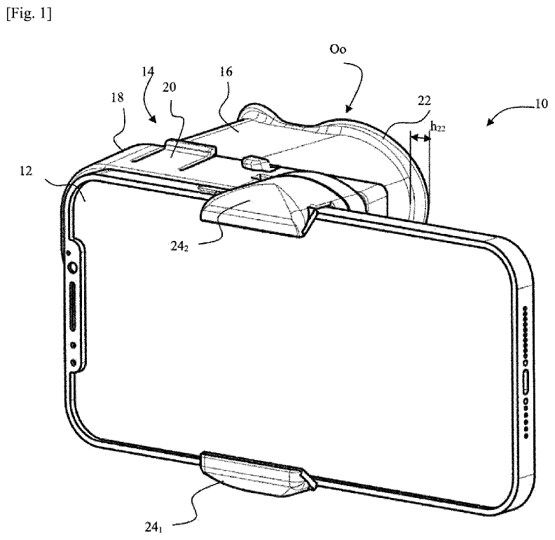

The portable scanner 6 can be integrated into a mobile telephone 12, as shown in FIG. 1, or be in communication with a mobile telephone. Step a) is therefore easy for the user to implement. The mobile telephone can also be used to transfer the updated model to a remote computer.

The updated instant can be during orthodontic treatment undergone by the user or outside orthodontic treatment.

In step a), the portable scanner is preferably hand-held by the user. Preferably, it is not immobilized, for example by means of a structure resting on the ground, such as a tripod. Preferably, the user's head is not immobilized.

In one embodiment, the user scans the dental arch without using any device other than the portable scanner.

In a preferred embodiment, the user uses a tool to free their lips, and better expose their dental arch to the portable scanner. The tool may be a spoon, for example, inserted into the mouth.

In one embodiment, the user uses a retractor and/or a mouth support which they partially insert into their mouth.

### Portable Scanner Support

In a particularly advantageous embodiment, in step a), the user uses a kit 10 comprising the portable scanner 6 and a support 14 (FIG. 1) which makes it possible to simultaneously
spread the user's lips to expose the teeth, and
facilitate the positioning and orientation of the portable scanner 6 in relation to the teeth.

The support 14 preferably has the general shape of a tubular body, one opening of which, known as the “oral opening” Oo, is intended to be introduced into the patient's mouth, and the opposite opening of which, known as the “acquisition opening”, faces the lens of the portable scanner, which is rigidly attached, preferably removably, to the support 14.

Preferably, the acquisition opening also faces a portable scanner flash, which can be used to illuminate the user's teeth during acquisition.

The support 14 makes it possible to define a spacing between the portable scanner and the oral opening Oo, as well as an orientation of the portable scanner relative to the oral opening. Advantageously, in the service position, the data acquired by the portable scanner 6 through its lens, the acquisition opening and the oral opening are thus acquired at a predetermined distance from the user's teeth and according to a predefined orientation. Preferably, the support is configured so that this spacing and orientation are constant.

Preferably, the support 14 comprises:
a tubular spacer 16 which defines the oral opening Oo and preferably comprises a radially outwardly extending flange 22 at the periphery of the oral opening Oo, to be inserted between the user's lips and teeth, and
an adapter 18 to which the portable scanner 6 is attached, for example clamped between two jaws 24₁ and 24₂, as shown in FIG. 1, the adapter 18 being rigidly attached to the spacer 16, preferably removably, for example by means of a clip 20, or integral with the retractor, so that the portable scanner lens can “see” the oral opening.

The maximum height h22 of rim 22 is preferably greater than 3 mm and less than 10 mm.

To acquire the acquired model, the user attaches the tubular spacer 16 to the adapter 18 by means of the clip 20, then attaches the portable scanner to the adapter 18 so that the portable scanner can scan through the tubular spacer 16 and the adapter 18. The user then introduces the end of the tubular spacer opposite the portable scanner into their mouth, inserting the rim 22 between their lips and teeth. In this way, the lips rest on the outside of the tubular spacer 16, providing a clear view of the teeth through the oral opening Oo.

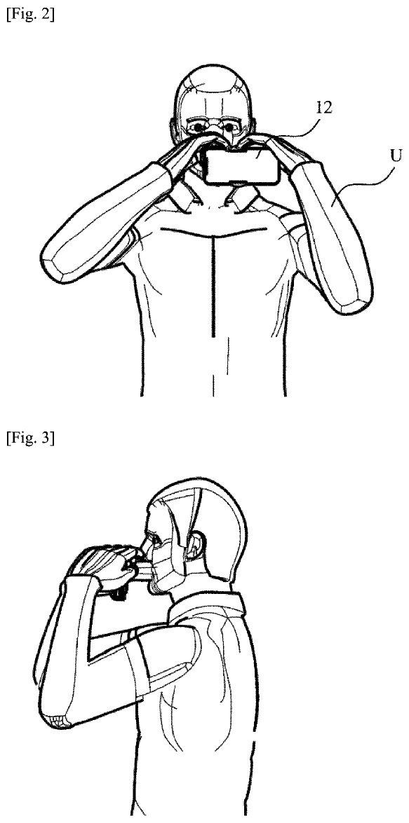

In the service position obtained, as shown in FIGS. 2 and 3, the teeth do not rest on the support, so that user U can, by turning the head relative to the support, modify the teeth that are visible to the portable scanner through the oral opening. The user can also change the spacing between their dental arches. In particular, the support separates the lips, but does not press on the teeth so as to move the two jaws apart.

The acquired model can represent one or both dental arches, in full or in part.

### Breaking Down the Acquired Model

In one embodiment, the arch model acquired with the portable scanner is broken down, preferably to define at least one tooth model 30. In one embodiment, the updated model is thus reduced to a portion of the acquired model, preferably reduced to a tooth model.

Preferably, steps b) and c) are then carried out successively for each tooth model.

Any known breakdown method can be used to break down a model.

Correcting the updated model, which may be derived from a breakdown of the acquired model, involves modifying it so that it is more in line with the object it models. To this end, the model's resolution can be improved and/or it can be added to and/or it can be given more realistic colors, for example to make it hyper-realistic, and/or it can be cleaned. Model cleaning consists of removing parts of the model that do not model the target object, for example by removing the representation of an orthodontic attachment when the target object is a tooth, or removing defects resulting from the acquisition operation, in particular to clean up artifacts due to saliva during acquisition.

### Correction

The updated model is preferably computer-processed for correction. The updated model can be corrected after or before simplification.

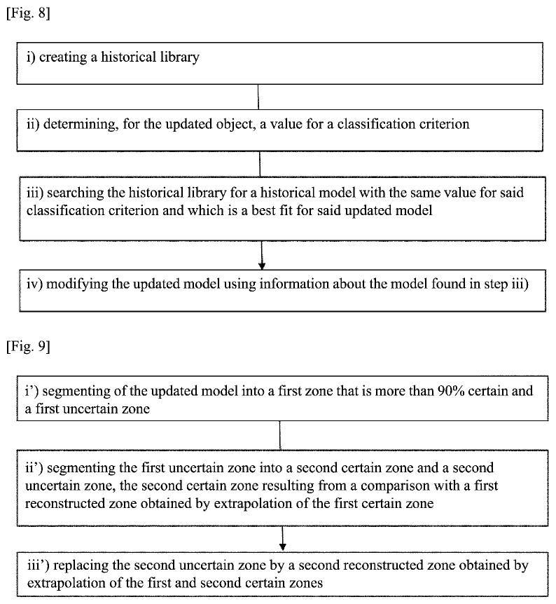

In a preferred embodiment, shown in FIG. 8, the updated model is compared with a “correction model”, then corrected according to the results of this comparison.

Preferably, the following steps are taken when the model to be corrected is a tooth model:
i) creating a historical library of over 1,000 tooth models, known as “historical tooth models”, and assigning each historical tooth model a tooth number;
ii) analyzing the tooth model to be corrected, so as to determine the tooth number modeled by said tooth model to be corrected;
iii) searching, in the historical library, for a historical tooth model with the same number and with maximum proximity to the tooth model to be corrected, or “optimal tooth model”;
iv) modifying the tooth model to be corrected on the basis of information on the optimal tooth model, which may comprise replacing the tooth model to be corrected with the optimal tooth model.

In step i), a historical library is created, preferably comprising more than 2,000, preferably more than 5,000, more preferably more than 10,000 and/or less than 1,000,0000 historical tooth models.

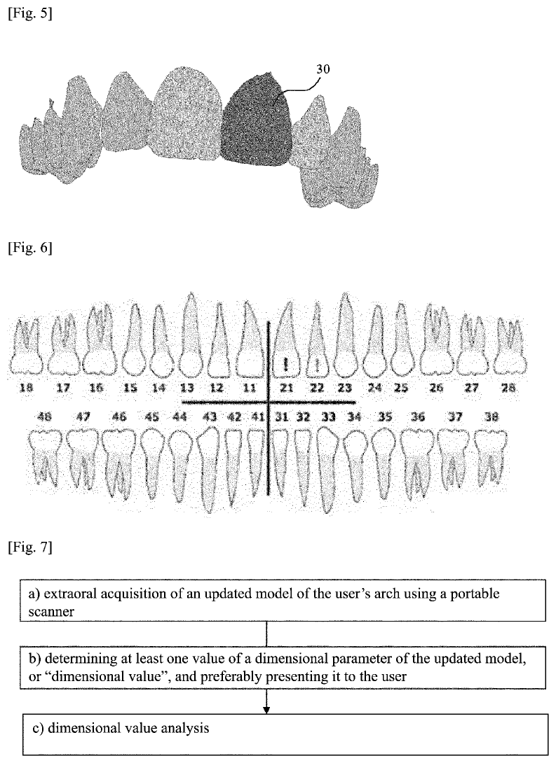

In particular, a historical tooth model can be obtained from a CT scan model of a “historical” patient's dental arch. This arch model can be cut to isolate tooth representations, that is, tooth models, as shown in FIG. 5.

The historical library therefore contains historical tooth models and the numbers of the teeth modeled by these historical tooth models.

In step ii), the tooth model to be corrected is analyzed to determine its number.

Tooth numbers are traditionally assigned according to a standard rule. Knowing this rule and the number of a model tooth is enough to determine the numbers of the other tooth models.

In a preferred embodiment, the shape of the tooth model to be corrected is analyzed to define its number. This shape recognition is preferably performed using a neural network.

Preferably, a neural network is used, preferably selected from the “Object Detection Networks”, for example from the following neural networks: R-CNN (2013), SSD (Single Shot MultiBox Detector: Object Detection network), Faster R-CNN (Faster Region-based Convolutional Network method: Object Detection network), Faster R-CNN (2015), SSD (2015), RCF (Richer Convolutional Features for Edge Detection) (2017), SPP-Net, 2014, OverFeat (Sermanet et al.), 2013, GoogleNet (Szegedy et al.), 2015, VGGNet (Simonyan and Zisserman), 2014, R-CNN (Girshick et al.), 2014, Fast R-CNN (Girshick et al.), 2015, ResNet (He et al.), 2016, Faster R-CNN (Ren et al.), 2016, FPN (Lin et al.), 2016, YOLO (Redmon et al.), 2016, SSD (Liu et al.), 2016, ResNet v2 (He et al.), 2016, R-FCN (Dai et al.), 2016, ResNext (Lin et al.), 2017, DenseNet (Huang et al.), 2017, DPN (Chen et al.), 2017, YOL09000 (Redmon and Farhadi), 2017, Hourglass (Newell et al.), 2016, MobileNet (Howard et al.), 2017, DCN (Dai et al.), 2017, RetinaNet (Lin et al.), 2017, Mask R-CNN (He et al.), 2017, RefineDet (Zhang et al.), 2018, Cascade RCNN (Cai et al.), 2018, NASNet (Zoph et al.), 2019, CornerNet (Law and Deng), 2018, FSAF (Zhu et al.), 2019, SENet (Hu et al.), 2018, ExtremeNet (Zhou et al.), 2019, NAS-FPN (Ghiasi et al.), 2019, Detnas (Chen et al.), 2019, FCOS (Tian et al.), 2019, CenterNet (Duan et al.), 2019, EfficientNet (Tan and Le), 2019, AlexNet (Krizhevsky et al.), 2012, Cbnet (2020), Point-gnn (2020), MDFN (2020), CADN (2021).

Preferably, the neural network is trained by providing tooth models as input and the associated tooth number as output. The neural network thus learns to provide a tooth number for a tooth model presented to it as input.

The tooth model to be corrected can then be modified from a historical tooth model with the same number.

In step iii), the historical tooth model having the same number as the tooth model to be corrected is searched in the historical library for the tooth model having the closest proximity to the tooth model to be corrected. This historical tooth model is referred to as the “optimal tooth model”.

“Proximity” is a measure of the difference in shape between the historical tooth model and the tooth model to be corrected. The difference in shape can be, for example, an average distance between the historical tooth model and the tooth model to be corrected after they have been arranged in a standardized configuration.

Preferably, maximum proximity, or “best fit”, is considered to be achieved when the cumulative Euclidean distance between the points of the historical tooth model and those of the tooth model to be corrected is minimal.

In step iv), the tooth model to be corrected is modified on the basis of information about the optimal tooth model, which serves as the correction model.

For example, those zones of the tooth model to be corrected which, in the normalized configuration, are more than 1 mm away from the optimum tooth model can be replaced by the zones of the optimum tooth model facing them, and/or the “blank” zones of the tooth model to be corrected, that is, undefined zones that face non-blank zones of the optimal tooth model, can be replaced by these zones of the optimal tooth model.

Modifying the tooth model to be corrected can also involve replacing the tooth model to be corrected with the optimal tooth model.

Preferably, steps i) to iv) are carried out for each tooth model cut from the acquired model.

The above procedure can be applied to an updated model of a dental arch. In steps ii) and iii), the classification criterion of the updated model is adapted accordingly. Instead of the tooth number, the classification criterion can be, for example, one or more attributes relating to an arch, such as arch width, or to both arches. The classification criterion can be chosen in particular from those listed above, in the definition of a classification criterion.

The updated model can be submitted to a neural network trained for this purpose by means of a training base. In particular, the neural network can be selected from the following networks: Shape Inpainting using 3D Generative Adversarial Network and Recurrent Convolutional Networks (2017), Deformable Shape Completion with Graph Convolutional Autoencoders (2018), Learning 3D Shape Completion Under Weak Supervision (2018), PCN: Point Completion Network (2019), TopNet: Structural Point Cloud Decoder (2019), RL-GAN-Net: A Reinforcement Learning Agent Controlled GAN Network for Real-Time Point Cloud Shape Completion (2019), Cascaded Refinement Network for Point Cloud Completion (2020), PF-Net: Point Fractal Network for 3D Point Cloud Completion (2020), Point Cloud Completion by Skip-attention Network with Hierarchical Folding (2020), GRNet: Gridding Residual Network for Dense Point Cloud Completion (2020), and Style-based Point Generator with Adversarial Rendering for Point Cloud Completion (2021).

For example, each record in the learning database may comprise:
an incomplete model of an object, for example
of a dental arch, or
of a tooth model, and
the same model, but complete.

Preferably, the objects modeled in the records belong to the same class defined by a classification criterion. For example, if these objects are teeth, the tooth number of tooth models is preferably identical for all records in the learning database.

Preferably, a neural network specializing in image generation is used, for example:
Cycle-Consistent Adversarial Networks (2017),
Augmented CycleGAN (2018),
Deep Photo Style Transfer (2017),
FastPhotoStyle (2018),
pix2pix (2017),
Style-Based Generator Architecture for GANs (2018),
SRGAN (2018),
WaveGAN 2020
GAN-LSTM 2019,
CSGAN 2021,
DivCo 2021.

After being trained with this learning base, the neural network can transform an incomplete model into a complete model by successively supplying it with the incomplete model for each record and the corresponding complete model as output.

The complete model serves as a “correction model”.

The correction model can be used to perform a quality check on the acquisition of the acquired model, that is, to check that this acquisition has not generated any defects. A defect is a part of the acquired model that does not correctly represent the dental arch(es). For example, the model may feature asperities or indentations that do not exist in reality, that is, on the dental arch(es).

The correction of the acquired model can also be used to remove such defects resulting from the acquisition operation.

### Cleaning

Preferably, the updated model is cleaned independently of the above modification method (steps i) to iv)). The aim is to process the updated model to remove the representation of an external object, and to replace it with a surface that represents as faithfully as possible the surface of the arch covered by this object.

In a preferred embodiment, shown in FIG. 9, the updated model is cleaned to remove the representation of an object external to the user, for example an orthodontic bracket, at least partially masking the object to be modeled, for example a tooth, by proceeding according to the following steps:
i′) defining:
a first certain zone made up of the points of the updated model representing the object to be modeled, for example a tooth, with an accuracy greater than 90%, or “first certain points”, and
a first uncertain zone, constituting the 100% complement of the updated model; ii′) extrapolation of the first certain zone, starting from the first certain zone only, to define, in the region of the first uncertain zone, a first reconstructed zone, then
defining:
a second certain zone made up of the points of the first uncertain zone separated from the first reconstructed zone by a distance less than a threshold distance, or “second certain points”; and
a second uncertain zone, constituting the 100% complement of the first uncertain zone;
iii′) extrapolating the set consisting of the first and second certain zones, from the single set, to define, in the region of the second uncertain zone, a second reconstructed zone, then
replacing the second uncertain zone with the second reconstructed zone, so as to obtain a cleaned updated model.

The advantage of these operations is that the representation of the external object is removed from the updated model, resulting in a cleaned updated model representing the object to be modeled with good accuracy.

The external object may be all or part of an orthodontic appliance, a crown, an implant, a bridge, an elastic band or a veneer. It can also be food, a drop of saliva, or all or part of a tool.

In step i′), the representation of the external object is isolated.

Specifically, we identify the points in the updated model that are almost certainly representations of points on the arch.

Algorithms for detecting objects in images are well known to the person skilled in the art. Preferably, a neural network is used, preferably selected from the “Object Detection Networks”, for example from the ones listed above.

After training, these neural networks are able to detect those points in the updated model which, with an accuracy threshold greater than or equal to 90%, represent points in the arch, or “first certain points”. All these points, known as the “first certain zone”, make up a fraction of the updated model. The points in the updated model that are not in the first certain zone collectively form the “first uncertain zone”.

Preferably, the accuracy threshold is greater than 95%, preferably greater than 98%, more preferably greater than 99% and/or less than 99.99%.

Training a neural network to detect an object in an image poses no difficulty for the person skilled in the art. For example, it can be supplied with arch models as input and the same arch models as output, on which zones representing the arch and zones representing an external object have been identified. It learns how to define these zones on an arch model.

The aim of the following steps is to fill in the “first blank zone” of the updated model, which appears when the first uncertain zone is removed.

In step ii′), the first certain zone is used to define a surface that fills said first blank zone. This zone is called the “first reconstructed zone”.

The techniques used to achieve this extrapolation are well known. Examples include WENDLAND, Holger. Piecewise polynomial, positive definite and compactly supported radial functions of minimal degree. Advances in Computational Mathematics, 1995, vol. 4, no I, p. 389-396.

To refine the reconstruction of the arch surface concealed by the external object, the points of the first uncertain zone that are close to the first reconstructed zone are then identified. These points are therefore points in the updated model that are close to a surface extrapolated from points representing, with virtual certainty, points on the arch.

These points in the updated model, or “second certain points”, are also considered to be, with a high degree of accuracy, points representing points on the arch. These points are known as the “second certain zone”. These points are therefore points in the updated model that the analysis in step i′) had discarded, but which are retained because they are close to a surface extrapolated from the points that the analysis in step i′) had retained.

The points in the updated model that don't belong to either the first certain zone or the second certain zone collectively form the “second uncertain zone”.

The proximity of a point in the first uncertain zone to the first reconstructed zone can be assessed by measuring the Euclidean distance between this point and the first reconstructed zone. A point in the first uncertain zone is considered to enter the second certain zone if this distance is less than a threshold distance.

If the model is to scale 1, that is, represents the modeled object with its actual dimensions, the threshold distance is preferably greater than 0.1 mm and/or less than 1 mm.

The threshold distance can also be determined by analyzing the distribution of said Euclidean distances between points in the first uncertain zone and the first reconstructed zone, for example as a function of the mean and standard deviation of these distances. A dynamic calculation using a method such as the “3 sigma rule” can be used, for example.

In step iii′), the aim is to replace the second uncertain zone with a second reconstructed zone that better matches the arch surface. To this end, the first and second certain zones are extrapolated into the region of the second uncertain zone.

Particularly remarkable is the fact that the extrapolation is not based on the first certain zone alone, but on the first and second certain zones together. Tests have shown that this extrapolation produces a second reconstructed zone representing the arch surface with a high degree of reliability.

The extrapolation in step iii′) can use the same methods as those used in step ii′). It can also use different methods.

The first and second certain zones and the second reconstructed zone constitute the updated, cleaned model, on which the representation of external objects has been removed.

### Appearance Correction

Preferably, the updated model is made hyperrealistic, preferably by means of a neural network.

The updated model can be submitted to a neural network trained for

this purpose by means of a learning base, as described for example in http://cs230.stanford.edu/projects_winter_2020/reports/32639841.pdf.

For example, each record in the learning database may comprise:
a raw model of an object, for example
of a dental arch, or
of a tooth model, and
the same model, but hyper-realistic.

Raw models are preferably similar in appearance to the updated model. They can be scans, preferably made with a scanner identical or similar to the portable scanner used in step a).

Raw models, for example, may have been rendered hyperrealistic by photo projection.

Preferably, the objects modeled in the records belong to the same class defined by a classification criterion. For example, if these objects are teeth, the tooth number of tooth models is preferably identical for all records in the learning database.

Preferably, a neural network specializing in image generation is used, for example:
Cycle-Consistent Adversarial Networks (2017)
Augmented CycleGAN (2018)
Deep Photo Style Transfer (2017)
FastPhotoStyle (2018)
pix2pix (2017)
Style-Based Generator Architecture for GANs (2018)
SRGAN (2018).

After being trained with the training base, providing it successively with the raw model as input and the hyperrealistic model as output for each record, the neural network can transform a raw model into a hyperrealistic model.

Thanks to the correction methods described above, an updated model can be advantageously transformed into an updated model representing the modeled object, for example the real arch, with a high degree of realism.

### Simplification

Before being used, for example in step b), the updated, possibly corrected model can be simplified, in particular to facilitate processing in step b). Simplification can also be carried out before or after any correction, or between two correction treatments.

The updated, preferably corrected, model is preferably displayed on a screen, preferably on the mobile telephone screen when the mobile phone incorporates the portable scanner and/or on a screen in a dental professional's office.

One or more of the breakdown and/or correction and/or cleaning and/or appearance correction and/or simplification operations described above can be carried out
in the portable scanner, preferably in the mobile telephone incorporating the portable scanner or in communication with an acquisition tool, or
in a data processing center in communication with said mobile telephone, to which said mobile telephone has transmitted the acquired or updated model.

In step b), at least one value of a dimensional parameter of the updated model, or “dimensional value”, and/or at least one value of an appearance parameter of the updated model, or “appearance value”, is determined.

Step b) can be implemented in the mobile telephone or in a processing center, remote from the mobile telephone, to which the mobile telephone transmits the updated model.

The updated model used in step b) can be
the acquired model, that is, the raw model as generated by the portable scanner, or
a part of the acquired model, for example resulting from a computer breakdown of the acquired model, or
said model acquired after correction and/or simplification, or
said part of the model acquired after correction and/or simplification.

A “dimensional value” is a value that depends on the shape of the updated model. This value is that of a “dimensional parameter”, which can be chosen from among
a dimension of the updated model, for example the width, length or height of the dental arch or a tooth;
a distance of a point in the updated model from a reference frame, or
a parameter derived from these dimensions and distances, e.g. an orthodontic index, a canine/molar occlusion class, an overbite or overjet measurement, a tooth number, or an indication of the presence or absence of a tooth.

The dimensional value can be measured on the updated model or obtained from one or more measurements made on the updated model.

For example, we can measure the distance between two teeth, the position of a noteworthy point in relation to a reference frame, e.g. orthonormal, fixed in relation to the actual object (arch or tooth in particular) or in relation to another tooth, e.g. to assess the alignment of a tooth in relation to other teeth, the misalignment of a tooth in relation to others or in relation to a predetermined position in the reference frame, the positioning of one or more teeth in relation to a fixed or removable orthodontic appliance positioned on the teeth or soft tissues, the index of crowding and/or irregularity of the arch, the misalignment of a tooth in relation to the other teeth or in relation to the gum, a deformation of a tooth, for example the depth of a cavity, a deformation of the gum, the width of the arch or the relative position of one arch in relation to the other.

The dimensional value can also be a measure of a difference in shape between the updated model and a reference model. In particular, tooth shapes and/or positions can be compared in the updated model and in a reference model.

An “appearance value” is a value that depends on the surface appearance of the updated model. This value is that of an “appearance parameter”, which can be chosen from among color, reflectance, transparency, reflectivity, hue, translucency, opalescence and an indication of the presence of tartar, dental plaque or food on the tooth.

The appearance value can also be a measure of a difference in shape between the updated model and a reference model. In particular, tooth appearances can be compared in the updated model and in a reference model.

The reference model is chosen according to the intended application.

For example, if the objective is to check whether the dental situation is normal at the updated instant, that is, to verify that it does not require the intervention of a dental care professional, particularly for therapeutic or aesthetic reasons, the reference model can be a model that represents an object of the same type as the updated object, or even the updated object, in a dental situation considered normal at the updated instant.

The reference model can be representative of a set of individuals, preferably comprising more than 100 individuals, preferably more than 1000 individuals and/or less than 1,000,0000 individuals, for example
a tooth from a typodont if the updated object is a tooth, or
an arch corresponding to an average arch shape for all individuals, if the updated object is an arch.

The reference model can be a model that represents an object of the same type as the updated object, preferably the updated object, but in a position and/or with a shape and/or with an appearance that is that/those of the updated object anticipated for a reference instant, prior to or subsequent to the updated instant or simultaneous with the updated instant.

In particular, the reference instant can be a stage of orthodontic treatment undergone by the user (e.g. at the beginning or end of orthodontic treatment, or at an intermediate stage of orthodontic treatment known as intermediate “set-up” or “staging”).

The time interval between the updated and reference instants can be greater than one week, preferably greater than 2 weeks, 4 weeks, 6 weeks, 2 months and/or less than 6 months.

The reference model can be obtained by means of a scanner, for example with the user's portable scanner, preferably by means of a professional scanner, or be obtained by construction from photos of the arch and a library of historical teeth, as described in EP18184486, equivalent to U.S. Pat. No. 16/031,172.

The reference model is preferably obtained by computer simulation, so that it represents the dental arch in the configuration expected at the reference instant, in particular at the end of orthodontic treatment or at the updated instant.

For example, it may result from a modification of an initial model, for example generated by means of a scan of a user's arch, preferably generated more than a week before the updated instant, for example at the start of orthodontic treatment. The initial model is traditional broken down to define tooth models. Moving the tooth models then simulates the orthodontic treatment process.

An example of software for manipulating tooth models and creating a treatment scenario is the program Treat, described at https://en.wikipedia.org/wiki/Clear_aligners#cite_note-invisalignsystem-10. U.S. Pat. No. 5,975,893A also describes the creation of a treatment scenario.

In one embodiment, the following is performed:
breaking down the reference model produced prior to the updated instant or of the updated model, to generate tooth models,
moving one or more of said tooth models, without deforming the tooth models, until a modified model is obtained which has a best fit with the updated or reference model, respectively,
an evaluation of the positional difference (determination of at least one dimensional value) of at least one tooth model between its position in the reference or updated model, respectively, and its position in the modified model.

In step c), the dimensional value and/or appearance value determined in step b) is/are used, in particular to decide whether action for therapeutic or aesthetic purposes is required and/or to help determine such action.

The dimensional value and/or appearance value, and preferably the updated model, can be presented to the user, for example by being displayed on the user's mobile telephone screen.

In addition or alternatively, they can also be transmitted, preferably over the air, preferably by a mobile telephone integrating the portable scanner or in communication with the acquisition tool, to a dental care professional, in particular an orthodontist, or to a remote computer in communication with the mobile telephone.

Preferably, the dimensional value and/or appearance value is/are interpreted, preferably by computer, preferably by a mobile telephone integrating the portable scanner, and a recommendation is presented to the user, preferably on the mobile telephone screen.

### Use of Up-to-Date Images

In a particularly advantageous embodiment, in step a) the user acquires one or more “updated” images, preferably extra-oral, in addition to the updated model. Preferably, the user uses the mobile telephone implemented to acquire the acquired model.

Preferably, the updated images are photographs or images taken from a video. They are preferably in full color, preferably in true color. Preferably, they depict the dental arches substantially as seen by the operator of the image acquisition device.

The information provided by updated images complements that provided by the acquired model. In particular, the information can relate to a dimension and/or the appearance of one or more objects, preferably teeth, represented in the updated image(s). In particular, the analysis of an updated image, preferably by computer, can be used to confirm and/or correct a dimensional value and/or an appearance value determined from the updated model, and/or to supplement the lessons learned from the updated model.

For example, the updated model may detect a cavity on the surface of a tooth, and an updated image may show a darker zone at the location of this cavity. The updated image confirms the presence of the cavity. It also allows you to confirm your position. By analyzing the model and updated images, it is possible to detect and monitor cavities.

Updated images can also reliably provide information on the appearance of teeth, such as their color. Projected onto the updated model, they allow the surface of the updated model to be colored in a highly realistic way.

Preferably, multiple updated images are acquired at different angles, that is, with different orientations of the acquisition device with respect to the user's oral cavity. For example, the updated image set may comprise 6 images representing the dental arches “front views”, “front-right views”, “right views”, “front-left views”, “left views” and “bottom views”, respectively.

Preferably, at least one updated image is acquired facing the user (front view). Preferably, at least one updated image is acquired from the user's right, and at least one updated image is acquired from the user's left.

The set of updated images preferably comprises more than two, preferably more than three, preferably more than 5, preferably more than 6 and/or less than 30, preferably less than 20, preferably less than 15, preferably less than 10 updated images.

In one embodiment, the updated images are processed to generate a so-called correction model and/or a so-called reference model. Any conventional techniques can be used for this purpose.

By acquiring two models at the updated instant, namely the updated model and a model obtained from updated images, and then comparing these models, it is possible to make the most of the 3D and 2D representations provided by the portable scanner and the image acquisition device, respectively.

The method can be implemented independently of any orthodontic treatment, in particular to monitor that the position and/or shape of teeth are not “abnormal”, that is, when they do not meet a therapeutic or aesthetic standard. Preferably, an appointment with a dental professional should then be made. The method can be implemented prior to orthodontic treatment.

Prior to orthodontic treatment, the method can be used, for example, to acquire the positioning and anatomy of the future patient's teeth and initiate the manufacture of an interceptive orthodontic appliance or a custom-made orthodontic appliance, such as transparent orthodontic aligners, or to design an individualized treatment using archwires and brackets.

The method can be implemented during orthodontic treatment, in particular to monitor progress, with step a) being implemented less than 3 months, less than 2 months, less than 1 month, less than 1 week, less than 2 days after the start of treatment, that is, after the fitting of an appliance designed to correct the positioning of the user's teeth, known as an “active retainer”.

During orthodontic treatment, the method can be implemented to acquire an updated model of the teeth and enable the fabrication of a new orthodontic appliance, for example an implant, an orthodontic aligner, or a vestibular orthodontic appliance.

Preferably, the updated model generated in step a) and/or the value(s) determined in step b) is/are forwarded to a dental professional to help establish a diagnosis.

The method can also be used after orthodontic treatment, to check that the positioning of the teeth does not change unfavorably (“recurrence”). Step a) is then preferably carried out less than 3 months, less than 2 months, less than 1 month, less than one week, less than 2 days after the end of treatment, that is, after fitting a passive retainer to hold the teeth in position.

The dimensional value is preferably used to
detect a recurrence, and/or
determine the rate of change of tooth positioning, and/or
optimize the appointment date with a dental professional, and/or
evaluate the effectiveness of an orthodontic treatment, and/or
evaluate the evolution of tooth positioning towards a reference model corresponding to a given tooth positioning, in particular an improved tooth positioning, and/or
a current orthodontic treatment, for example by manufacturing a new series of orthodontic aligners, and/or
in a dental office, and/or
visualize and/or measure and/or detect dental plaque, and/or a cavity, and/or microfissures, and/or wear, for example resulting from bruxism or the use of active or passive orthodontic appliances, particularly in the event of breakage or detachment of an orthodontic arch;
visualize and/or measure and/or detect a change in volume, in particular during tooth growth or following an intervention by a dental professional, e.g. a deposit of glue on the surface of the teeth;
assess the need for interceptive treatment, prior to any orthodontic treatment, in particular to evaluate the benefits of orthodontic treatment.

The appearance value is preferably used to detect or evaluate a position or shape of a stain or cavity.

In a particularly advantageous embodiment, both the dimensional value and the appearance value are used. Advantageously, the method can thus be used for precise, localized monitoring of the evolution of certain pathologies, in particular stains, demineralization, or cavities.

As will now become clear, the invention provides a method enabling a particular user, for example a patient, to generate a model of one or more of their dental arches, or one or more of their teeth. They don't need any special equipment, apart from the portable scanner, which is preferably integrated into his mobile telephone.

The acquired model can be acquired without inserting the portable scanner into the user's mouth, that is, extra-orally. In particular, processing the updated model to correct it allows it to be corrected to model regions of the mouth to which the portable scanner did not have access, for example in an interproximal space.

In one embodiment, in step a), the acquired model is coarse. In particular, it can represent a “3D skeleton” of the user's dental arch(es), with less than 500 points, less than 200 points, less than 100 points or less than 50 points and/or more than 10 points. Processing the updated model for correction, in particular with a neural network or from a historical library, can advantageously reconstitute a much more accurate model of the user's dental arch(es).

In one embodiment, the portable scanner is partially inserted into the user's mouth. Advantageously, the lingual surfaces of the teeth can be scanned.

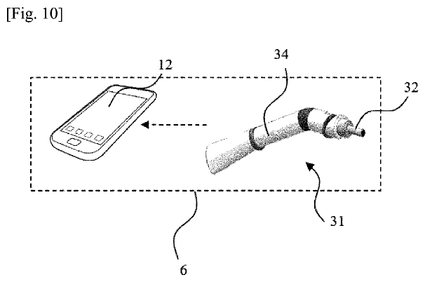

As shown in FIG. 10, the portable scanner 6 preferably comprises a mobile telephone 12 and an acquisition tool 31 in communication with the mobile telephone, preferably over the air, preferably via Bluetooth®. Cable communication is also possible.

The acquisition tool is equipped with an acquisition head 32 that can be inserted into the user's mouth. The acquisition head acquires the acquired model and transmits it to the mobile telephone 12, or acquires a signal, for example a set of images, and transfers it to the mobile telephone 12 so that the latter generates the acquired model from said signal.

Preferably, the acquisition tool has no physical link to the mobile telephone or is connected to the mobile telephone by a flexible link, such as a cable.

Preferably, the acquisition tool has a handle 34 to facilitate handling, by the user directly or someone close by, for example in the manner of a toothbrush.

In one embodiment, the acquisition tool is attached to the mobile telephone, for example by means of a clip, a hook-and-loop fastener, clamping jaws, a screw, a magnet, a cover or a flexible, preferably elastic, band. Attachment can also be achieved by complementing the shape of the mobile telephone. For example, the acquisition tool can be attached to a telephone case.

In one embodiment, the method also uses a measuring head in communication with the mobile telephone, which is inserted into the user's mouth in order to acquire additional data, e.g. data on
the space between teeth
the lingual surfaces of the teeth
the palate, including, for example, the median palatine suture
the soft tissues (canker sores, benign or malignant lesions, recessions, etc.)
the tooth shade
the presence of cavities or stains
the condition and/or shape of implants, crowns and/or bridges
the condition of vestibular or lingual treatment appliances (e.g. lingual or vestibular brackets, palatal expander, or any other treatment aid) or retention devices (palatal arch)
distances between different parts of the same vestibular, lingual or other auxiliary equipment
condition of anchoring devices (mini-screws)
distance between anchoring devices and appliances in the mouth of soft-tissue stitches
post-surgical soft tissue healing
the curve of Spee
the curve of Wilson
the intercanine distance
the intermolar distance

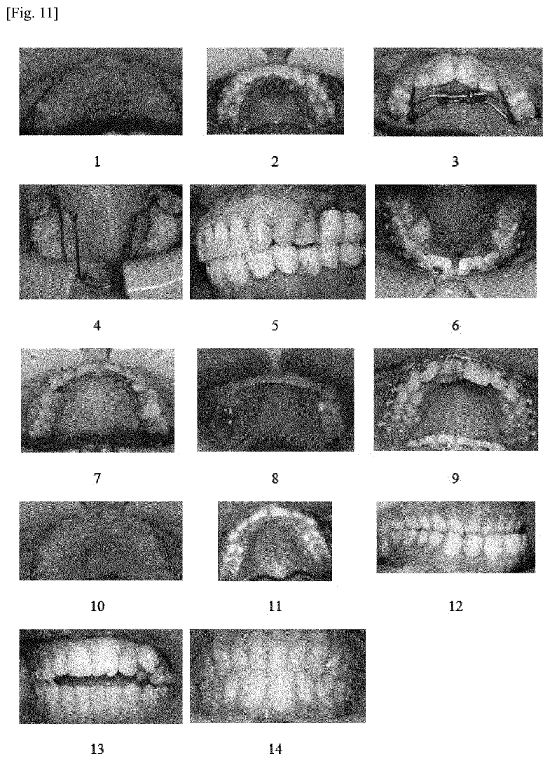

FIG. 11 shows various images providing additional data, in particular on the palate, including a median palatine suture (image 1), soft tissue sutures (image 2), distances between different parts of the same vestibular appliance, lingual or other auxiliary appliances (images 3 and 4), the condition and/or shape of implants, crowns and/or bridges (images 5 and 8), the condition of anchorage devices (mini-screws) and the distance between anchorage devices and appliances present in the mouth (image 6), the condition of vestibular or lingual treatment appliances (e.g. lingual brackets, vestibular brackets, maxillary circuit breaker or other treatment aids) or retainers (palatal arch) (image 7), interdental space and post-surgical soft-tissue healing (image 9), lingual surfaces of teeth (image 10), intercanine distance and intermolar distance (image 10), tooth shade (image 11), curve of Spee (image 12), curve of Wilson (image 13), and presence of cavities or stains (image 14).

The measuring head can be integrated into a measuring tool with one or more of the features of the acquisition tool. Unlike the latter, however, the measuring tool is not used to acquire the acquired model.

The acquired model can then be corrected, in particular for completion and/or cleaning and/or hyperrealism. The user can transmit a model to a dental professional, dental professional whom they possibly have never met, which the dental professional can analyze, in particular to establish a diagnosis and/or to give advice to the user and/or to set an appointment date.

Of course, the invention is not limited to the embodiments described and shown.

The methods for correcting and simplifying the updated model described above are inventions, independently of the description method.

### Further Development

In addition to the method described above and more generally, the invention also relates to a method for acquiring at least one image of at least one dental arch of a user by means of a mobile telephone and an acquisition tool comprising an acquisition head provided with a camera, preferably suitable for insertion into the user's mouth, wherein method the acquisition head:
acquires said image and transmits it to the mobile telephone, or - acquires a signal and transfers it to the mobile telephone so that said mobile telephone generates the image from said signal, either autonomously or with the help of a computer with which said mobile telephone is in communication.

Said at least one image is preferably a photo, preferably a photo depicting the dental arch realistically, as a person would observe it directly.

The image can be used to generate a model as per step a), but the image acquisition method according to the invention is no longer limited to this particular embodiment, as the image can be used for other purposes. This method is therefore referred to below as a “generalized method”.

Insofar as a feature described above for step a) is technically compatible with the generalized method, it can nevertheless be applied to this method.

The mobile telephone and the acquisition tool are preferably handled exclusively by the user.

Acquisition can be carried out extraorally, with the acquisition tool's camera not penetrating into the user's mouth. Acquisition can be carried out intraorally, with the acquisition tool's camera penetrating into the user's mouth.

In one embodiment, the acquisition tool is attached to the mobile telephone, for example by means of a clip, a hook-and-loop fastener, clamping jaws, a screw, a magnet, a cover or a flexible, preferably elastic, band. Attachment can also be achieved by complementing the shape of the mobile telephone. For example, the acquisition tool can be attached to a telephone case.

Preferably, however, the mobile telephone and the acquisition tool communicate with each other, but can be moved independently of each other. Preferably, no rigid device, preferably no mechanism, connects the mobile telephone and the acquisition tool, so that the mobile telephone can be moved in space, preferably in all spatial dimensions, without necessarily dragging the acquisition tool with it.

Preferably, the screen displays the scene observed by the acquisition head camera.

In particular, the independence of movement between the mobile telephone and the acquisition tool means that the mobile telephone screen can be used to view the scene observed by the acquisition head camera, without this viewing being hindered by the handling of the acquisition head.

In one embodiment, during acquisition, the user observes the mobile telephone screen, with the mobile telephone preferably stationary relative to the ground, for example on a table, and manipulates the acquisition tool. This makes it easy to position the acquisition tool in a desired position, preferably for extraoral acquisition. In addition, this embodiment allows the user to use the mobile telephone camera on the opposite side of the screen, without having to use a mirror.

Preferably, the user acquires at least one image seen from the front, preferably at least one image from the user's right, and even more preferably at least one image from the user's left.

Preferably, the user acquires at least one open-mouth image and at least one closed-mouth image.

The set of acquired images preferably comprises more than two, preferably more than three, preferably more than 5, preferably more than 6 and/or less than 30, preferably less than 20, preferably less than 15, preferably less than 10 acquired images.

Preferably, the user uses a tool to move away their lips, and better expose the dental arch to the camera of the acquisition tool. The tool may be a spoon, for example, inserted into the mouth.

In one embodiment, the user uses a retractor which they partially insert into their mouth.

Preferably, the generalized method comprises, after said acquisition, an analysis of said image in order to define the user's dental situation, and preferably to design an active or passive orthodontic treatment plan, and/or to check the proper implementation of an ongoing active or passive orthodontic treatment.

Preferably, the acquisition method involves, after said image analysis, manufacturing an orthodontic appliance, for example an orthodontic aligner, and preferably sending said orthodontic appliance to the user.

The uses mentioned above for updated images can also be applied to the image(s) acquired using the generalized method.

Said at least one image is preferably used to
detect a recurrence, and/or
determine the rate of change of tooth positioning, and/or
optimize the appointment date with a dental professional, and/or
evaluate the effectiveness of an orthodontic treatment, and/or
evaluate the evolution of tooth positioning towards a reference model corresponding to a given tooth positioning, in particular an improved tooth positioning, and/or
modify a current orthodontic treatment, for example by manufacturing a new series of orthodontic aligners, and/or
in a dental office, and/or
visualize and/or measure and/or detect dental plaque, and/or a cavity, and/or microfissures, and/or wear, for example resulting from bruxism or the use of active or passive orthodontic appliances, particularly in the event of breakage or detachment of an orthodontic arch;
visualize and/or measure and/or detect a change in volume, in particular during tooth growth or following an intervention by a dental professional, e.g. a deposit of glue on the surface of the teeth;
assess the need for interceptive treatment, prior to any orthodontic treatment, in particular to evaluate the benefits of orthodontic treatment.

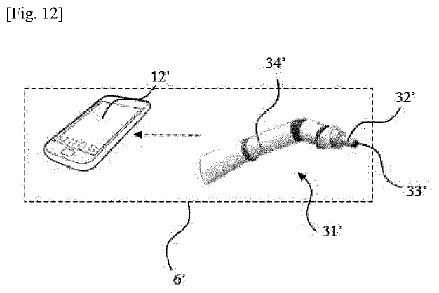

FIG. 12 shows a device 6′ for implementing such an image acquisition method. This kit comprises a mobile telephone 12′ and an acquisition tool 31′ in communication with the mobile telephone, preferably over the air, preferably by Bluetooth® or WiFi. Cable communication is also possible.

The acquisition tool 31′ is equipped with an acquisition head 32′ that can be inserted into the user's mouth. The acquisition head includes a camera 33′ that acquires the image and transmits it to the mobile telephone 12′, or acquires a signal and transfers it to the mobile telephone 12′ so that the latter can generate the image from said signal.

Preferably, the acquisition tool has no physical link to the mobile telephone or is connected to the mobile telephone by a flexible link, such as a cable.

Preferably, the acquisition tool has a handle 34′ to facilitate handling, by the user directly or someone close by, for example in the manner of a toothbrush.

The mobile telephone 12′ may include one or more of the features of the mobile telephone 12. Preferably, it is not attached to any support, and in particular to no support attached to the user such as the support 10 described above, and the user can manipulate it freely.

1. A method for acquiring at least one image of at least one dental arch of a user (U) by means of a mobile telephone (12′) and an acquisition tool (31′) comprising an acquisition head (32′) provided with a camera (33′), wherein method the acquisition head:
acquires said image and transmits it to the mobile telephone, or
acquires a signal and transfers it to the mobile telephone so that said mobile telephone generates the image from said signal, either autonomously or with the help of a computer with which said mobile telephone is in communication,
said at least one image being a photograph or an image extracted from a video.

2. The method according to the preceding claim, wherein the mobile telephone (12′) and the acquisition tool (31′) are handled exclusively by the user.

3. The method according to any of the preceding claims, wherein the acquisition is performed extraorally, the camera of the acquisition tool not penetrating into the user's mouth.

4. The method according to any of the claims 1 to 2, wherein the acquisition is performed intraorally, the camera of the acquisition tool penetrating into the user's mouth.

5. The method according to any of the preceding claims, wherein the mobile telephone and the acquisition tool can be moved independently of each other.

6. The method according to any of the preceding claims, wherein, during acquisition, the user observes the mobile telephone screen to view the scene observed by the acquisition head camera.

7. The method according to the immediately preceding claim, wherein during acquisition, the mobile telephone is stationary relative to the ground and the user handles the acquisition tool.

8. The method according to any of the preceding claims, wherein the user acquires at least one image seen from the front, at least one image from the user's right, at least one image from the user's left, at least one open-mouth image and at least one closed-mouth image.

9. The method according to any of the preceding claims, wherein the user uses a tool to move away their lips and better expose the dental arch to the camera of the acquisition tool.

10. The method according to the immediately preceding claim, wherein said tool is a retractor.

11. The method according to any of the preceding claims, wherein the acquisition tool is in communication with the mobile telephone by radio.

12. The method according to any one of the preceding claims, wherein said at least one image is used to
determine the rate of change of tooth positioning, and/or
optimize the appointment date with a dental professional, and/or
evaluate the change in tooth positioning towards a reference model corresponding to a given tooth positioning, and/or
visualize and/or measure and/or detect a microfissure, and/or wear, and/or
visualize and/or measure and/or detect a change in volume during tooth growth or following an intervention by a dental professional.

13. The method according to any of the preceding claims, wherein the image is used to generate a digital three-dimensional model.
